(19) United States
(12) Patent Application Publication
(10) Pub. No.: US 2025/0281183 A1
(43) Pub. Date: Sep. 11, 2025

(54) Left Atrial Appendage Occluder Devices

(71) Applicant: St. Jude Medical, Cardiology Division, Inc., St. Paul, MN (US)

(72) Inventors: Gary Erzberger, Corcoran, MN (US); Sadie Cronin, Plymouth, MN (US); Matthew Arthur Tanner, Minneapolis, MN (US); Evan Leingang, Plymouth, MN (US); Andrey Kai, Plymouth, MN (US); Sarah Grenell, Watertown, MN (US)

(73) Assignee: St. Jude Medical, Cardiology Division, Inc., St. Paul, MN (US)

(21) Appl. No.: 18/983,454

(22) Filed: Dec. 17, 2024

Related U.S. Application Data
(60) Provisional application No. 63/562,342, filed on Mar. 7, 2024.
(60) Provisional application No. 63/645,351, filed on May 10, 2024.
(60) Provisional application No. 63/694,087, filed on Sep. 12, 2024.

(51) Int. Cl. A61B 17/12

(52) U.S. Cl. CPC A61B 17/12122; A61B 17/1214

(57) ABSTRACT

A left atrial appendage (“LAA”) occluder may include a proximal disk configured to cover an ostium of the LAA, a distal lobe configured to be received within a cavity of the LAA, and a connecting member connecting the proximal disk to the distal lobe. The occluder may include first and second groups of stabilizing members having first and second engagement features, respectively, configured to engage tissue surrounding the LAA when the occluder is implanted. In an unbiased condition of the occluder, the distal lobe may have an axial length extending between a proximal face and a distal face of the distal lobe. The second engagement features may be positioned at least 33% of the axial length beyond the proximal face of the distal lobe, and the first engagement features may be positioned distal to the second engagement features.

### CROSS-REFERENCE TO RELATED APPLICATIONS

This application claims priority to the filing dates of (i) U.S. Provisional Patent Application No. 63/694,087, filed Sep. 12, 2024, (ii) U.S. Provisional Patent Application No. 63/645,351, filed May 10, 2024, and (iii) U.S. Provisional Patent Application No. 63/562,342, filed Mar. 7, 2024, the disclosures of all of which are hereby incorporated by reference herein.

### FIELD OF THE DISCLOSURE

The present disclosure relates generally to medical devices that are used in the human body. In particular, the present disclosure is directed to an occlusion device having a configuration that allows for more consistent and stable anchoring of the occlusion device within a tissue cavity. More specifically, the present disclosure is directed to an occlusion device with one or more rows of hooks or stabilizing wires that increase the resilience of the device to embolization or motion in the implanted condition.

### BACKGROUND

An occluder is a medical device used to treat (e.g., occlude) tissue at a target site within the human body, such as an abnormality, a vessel, an organ, an opening, a chamber, a channel, a hole, a cavity, a lumen, or the like. For example, an occluder may be used for Left Atrial Appendage (“LAA”) closures. An LAA is a normal anatomical structure which is a sac or pouch in the muscle wall of the left atrium. When a patient experiences atrial fibrillation (“AFib”), a blood clot may be formed within the LAA which may become dislodged and enter into the blood stream. By occluding the LAA, the release of blood clots from the LAA may be significantly reduced, if not eliminated. Various techniques have been developed to occlude the LAA. For instance, balloon-like devices have been developed that are configured to be implanted completely within the cavity of the LAA, while surgical techniques have also been developed where the cavity of the LAA is inverted and surgically closed.

Despite these techniques, it would be advantageous to provide an improved occlusion device that offers a reduced risk of adverse events such as embolization.

### SUMMARY OF THE DISCLOSURE

According to one aspect of the disclosure, a medical device is for occluding a left atrial appendage (“LAA”). The medical device may include a proximal disk configured to cover an ostium of the LAA in an implanted condition of the medical device, a distal lobe configured to be received within a cavity of the LAA in the implanted condition of the medical device, and a connecting member connecting the proximal disk to the distal lobe. The medical device may include a first group of stabilizing wires, each stabilizing wire in the first group having a first hooked end configured to engage tissue surrounding the LAA in the implanted condition of the medical device. The medical device may include a second group of stabilizing wires, each stabilizing wire in the second group having a second hooked end configured to engage tissue surrounding the LAA in the implanted condition of the medical device. In an unbiased condition of the medical device, the distal lobe may have an axial length extending between a proximal face and a distal face of the distal lobe. The second hooked ends of the stabilizing wires in the second group may be positioned at least one-third of the length beyond the proximal face of the distal lobe. The first hooked ends of the stabilizing wires in the first group may be positioned distal to the second hooked ends of the stabilizing wires in the second group. The second hooked ends of the stabilizing wires in the second group may be positioned between about 33% and about 55% of the axial length beyond the proximal face of the distal lobe. The axial length may be about 7.5 mm, and the second hooked ends of the stabilizing wires in the second group may be positioned between about 3.0 mm and about 4.0 mm beyond the proximal face of the distal lobe. The axial length may be about 10 mm, and the second hooked ends of the stabilizing wires in the second group may be positioned between about 3.75 mm and about 5.25 mm beyond the proximal face of the distal lobe. Each stabilizing wire in the first group may be positioned between a pair of stabilizing wires in the second group. Each stabilizing wire in the first group may be substantially identical in structure to each stabilizing wire in the second group. Each stabilizing wire in the first group may have an axial length that is equal to or longer than an axial length of each stabilizing wire in the second group. A total number of the stabilizing wires in the first group may be equal to a total number of stabilizing wires in the second group. A spacing between each adjacent pair of stabilizing wires may be substantially equal. A radial spacing between each adjacent pair of stabilizing wires may be between about 20 degrees and about 65 degrees.

According to another aspect of the disclosure, a medical device is for occluding a left atrial appendage (“LAA”). The medical device may include a proximal disk configured to cover an ostium of the LAA in an implanted condition of the medical device, a distal lobe configured to be received within a cavity of the LAA in the implanted condition of the medical device, a connecting member connecting the proximal disk to the distal lobe, and a plurality of stabilizing wires. The plurality of stabilizing wires may include a first group of hooked ends configured to engage tissue surrounding the LAA in the implanted condition of the medical device and a second group of hooked ends configured to engage tissue surrounding the LAA in the implanted condition of the medical device. In an unbiased condition of the medical device, the distal lobe may have an axial length extending between a proximal face and a distal face of the distal lobe, and the second group of hooked ends of the plurality of stabilizing wires may be positioned at least 33% of the axial length beyond the proximal face of the distal lobe. The first group of hooked ends of the plurality of stabilizing wires may be positioned distal to the second group of hooked ends of the plurality of stabilizing wires. The plurality of stabilizing wires may include a first group of stabilizing wires, and a second group of stabilizing wires. The first group of hooked ends may be positioned on the first group of stabilizing wires, the second group of hooked ends may be positioned on the second group of stabilizing wires. Each of the plurality of stabilizing wires may include at least one hooked end of the first group of hooked ends, and at least one hooked end of the second group of hooked ends. Each of the plurality of stabilizing wires may include one pair of hooked ends of the first group of hooked ends, and one pair of hooked ends of the second group of hooked ends. Each of the plurality of stabilizing wires may be isolated or separate from the other stabilizing wires of the plurality of stabilizing wires. Each group of stabilizing wires may comprise more than one stabilizing wire. The plurality of stabilizing wires may be coupled to, or connected to, the distal lobe. Each of the plurality of stabilizing wires may include a backing portion. Each backing portion may be U-shaped. Each backing portion may comprise one or more eyelets. A pair of eyelets may be provided for the respective pair of hooked ends of each backing portion. The eyelets of the plurality of stabilizing wires may be coupled to, or connected to, the distal lobe. Each of the plurality of stabilizing wires may include a backing portion, the backing portion including two legs extending therefrom, each of the two legs extending to a terminal end, the terminal end of each leg forming one of the pair of hooked ends of the first group of hooked ends, each of the pair of hooked ends of the second group of hooked ends extending from a respective one of the two legs. Each of the plurality of stabilizing wires may include a single hooked end of the first group of hooked ends, and a single hooked end of the second group of hooked ends. Each of the plurality of stabilizing wires may include a backing portion, the backing portion including two legs extending therefrom, a first one of the two legs terminating in the single hooked end of the first group of hooked ends, and a second one of the two legs terminating in the single hooked end of the second group of hooked ends, the first one of the two legs having a length that is longer than a length of the second one of the two legs. Each of the plurality of stabilizing wires may include a pair of hooked ends of the first group of hooked ends, and a single hooked end of the second group of hooked ends. Each of the plurality of stabilizing wires may include a backing portion, the backing portion including two legs extending therefrom, each of the two legs extending to a terminal end, the terminal end of each leg forming one of the pair of hooked ends of the first group of hooked ends, the single hooked end of the second group of hooked ends being formed integrally with the backing portion. Each of the plurality of stabilizing wires may include a backing portion, the backing portion including two legs extending therefrom, each of the two legs extending to a terminal end, the terminal end of each leg forming one of the pair of hooked ends of the first group of hooked ends, the single hooked end of the second group of hooked ends being coupled, via a connector, to the backing portion.

According to another aspect of the disclosure, a medical device is for occluding a left atrial appendage (“LAA”). The medical device may include a proximal disk configured to cover an ostium of the LAA in an implanted condition of the medical device, a distal lobe configured to be received within a cavity of the LAA in the implanted condition of the medical device, and a connecting member connecting the proximal disk to the distal lobe. The medical device may also include a first group of stabilizing wires, each stabilizing wire in the first group having a first hooked end configured to engage tissue surrounding the LAA in the implanted condition of the medical device. The medical device may also include a second group of stabilizing wires, each stabilizing wire in the second group having a second hooked end configured to engage tissue surrounding the LAA in the implanted condition of the medical device. The medical device may also include a third group of stabilizing wires, each stabilizing wire in the third group having a third hooked end configured to engage tissue surrounding the LAA in the implanted condition of the medical device. In an unbiased condition of the medical device, the distal lobe may have an axial length extending between a proximal face and a distal face of the distal lobe. The first hooked ends may be positioned at a first distance along the axial length. The second hooked ends may be positioned at a second distance along the axial length. The third hooked ends may be positioned at a third distance along the axial length. The first distance may be different than the second distance. The second distance may be different than the third distance. The third distance may be different than the first distance. The axial length may be about 10 mm. The first distance may be between about 8 mm and about 10 mm, such that the first hooked ends are positioned between about 0 mm and about 2 mm proximally of the distal face of the distal lobe. The second distance may be between about 3.3 mm and about 4.3 mm. The third distance may be between about 4.4 mm and about 5.4 mm. The medical device may include a total number of stabilizing wires in the first group that is at least twice as many as a total number of stabilizing wires in the second group and that is at least twice as many as a total number of stabilizing wires in the third group. A single stabilizing wire in either the second group or the third group may be positioned between each circumferentially adjacent pair of stabilizing wires in the first group. Each stabilizing wire may be separate from the other stabilizing wires. Each group of stabilizing wires may comprise more than one stabilizing wire. Each stabilizing wire may be connected to the distal lobe.

According to a further aspect of the disclosure there is provided a medical device for occluding a left atrial appendage (“LAA”), the medical device comprising: a proximal disk configured to cover an ostium of the LAA in an implanted condition of the medical device; a distal lobe configured to be received within a cavity of the LAA in the implanted condition of the medical device; a connecting member connecting the proximal disk to the distal lobe; and a plurality of stabilizing wires, the plurality of stabilizing wires including a first group of hooked ends configured to engage tissue surrounding the LAA in the implanted condition of the medical device and a second group of hooked ends configured to engage tissue surrounding the LAA in the implanted condition of the medical device, wherein in an unbiased condition of the medical device, the distal lobe has an axial length extending between a proximal face and a distal face of the distal lobe, the first group of hooked ends of the plurality of stabilizing wires being positioned distal to the second group of hooked ends of the plurality of stabilizing wires. The second group of hooked ends of the plurality of stabilizing wires may be positioned at least 33% of the axial length beyond the proximal face of the distal lobe. The plurality of stabilizing wires may comprise a first group of stabilizing wires and a second group of stabilizing wires. Each stabilizing wire in the first group of stabilizing wires may have a hooked end to provide the first group of hooked ends. Each stabilizing wire in the second group of stabilizing wires may have a hooked end to provide the second group of hooked ends. The plurality of stabilizing wires may comprise a third group of stabilizing wires. Each stabilizing wire in the third group may have a third hooked end configured to engage tissue surrounding the LAA in the implanted condition of the medical device. The first hooked ends may be positioned at a first distance along the axial length, the second hooked ends may be positioned at a second distance along the axial length, and the third hooked ends may be positioned at a third distance along the axial length. The first distance may be different than the second distance, the second distance may be different than the third distance, and the third distance may be different than the first distance.

According to a further aspect of the disclosure there is provided a medical device comprising a lobe and a plurality of stabilizing wires that are connected to the lobe, the plurality of stabilizing wires including a first group of hooked ends configured to engage tissue surrounding the lobe in an implanted condition of the medical device and a second group of hooked ends configured to engage tissue surrounding the lobe in the implanted condition of the medical device, wherein, in an unbiased condition of the medical device, the lobe has an axial length extending between a proximal face and a distal face of the distal lobe, and wherein the first group of hooked ends of the plurality of stabilizing wires are positioned distal to the second group of hooked ends of the plurality of stabilizing wires.

According to one aspect of the disclosure, a medical device for occluding a left atrial appendage (“LAA”) includes a proximal disk configured to cover an ostium of the LAA in an implanted condition of the medical device, a distal lobe configured to be received within a cavity of the LAA in the implanted condition of the medical device, and a connecting member connecting the proximal disk to the distal lobe. The medical device includes a first group of stabilizing members, each stabilizing member in the first group having a first engagement feature configured to engage tissue surrounding the LAA in the implanted condition of the medical device. The medical device includes a second group of stabilizing members, each stabilizing member in the second group having a second engagement feature configured to engage tissue surrounding the LAA in the implanted condition of the medical device. In an unbiased condition of the medical device, the distal lobe has an axial length extending between a proximal face and a distal face of the distal lobe, the second engagement features of the stabilizing members in the second group being positioned at least 33% of the axial length beyond the proximal face of the distal lobe, the first engagement features of the stabilizing members in the first group being positioned distal to the second engagement features of the stabilizing members in the second group. The second engagement features of the stabilizing members in the second group may be positioned between about 33% and about 55% of the axial length beyond the proximal face of the distal lobe. The axial length may be about 7.5 mm, and the second engagement features of the stabilizing members in the second group may be positioned between about 3.0 mm and about 4.0 mm beyond the proximal face of the distal lobe. The axial length may be about 10 mm, and the second engagement features of the stabilizing members in the second group may be positioned between about 3.75 mm and about 5.25 mm beyond the proximal face of the distal lobe. Each stabilizing member in the first group may be positioned between a pair of stabilizing members in the second group. Each stabilizing member in the first group may be substantially identical in structure to each stabilizing member in the second group. Each stabilizing member in the first group may have an axial length that is equal to or longer than an axial length of each stabilizing member in the second group. A total number of the stabilizing members in the first group may be equal to a total number of stabilizing members in the second group. A spacing between each adjacent pair of stabilizing members may be substantially equal. A radial spacing between each adjacent pair of stabilizing members may be between about 20 degrees and about 65 degrees.

According to another aspect of the disclosure, a medical device for occluding a left atrial appendage (“LAA”) includes a proximal disk configured to cover an ostium of the LAA in an implanted condition of the medical device, a distal lobe configured to be received within a cavity of the LAA in the implanted condition of the medical device, and a connecting member connecting the proximal disk to the distal lobe. The medical device includes a plurality of stabilizing members, the plurality of stabilizing members including a first group of engagement features configured to engage tissue surrounding the LAA in the implanted condition of the medical device and a second group of engagement features configured to engage tissue surrounding the LAA in the implanted condition of the medical device. In an unbiased condition of the medical device, the distal lobe has an axial length extending between a proximal face and a distal face of the distal lobe, the second group of engagement features of the plurality of stabilizing members being positioned at least 33% of the axial length beyond the proximal face of the distal lobe, the first group of engagement features of the plurality of stabilizing members being positioned distal to the second group of engagement features of the plurality of stabilizing members. The plurality of stabilizing members may include a first group of stabilizing members, and a second group of stabilizing members, the first group of engagement features being positioned on the first group of stabilizing members, the second group of engagement features being positioned on the second group of stabilizing members. Each of the plurality of stabilizing members may include at least one engagement feature of the first group of engagement features, and at least one engagement feature of the second group of engagement features. Each of the plurality of stabilizing members may include one pair of engagement features of the first group of engagement features, and one pair of engagement features of the second group of engagement features. Each of the plurality of stabilizing members may be isolated or separate from the other stabilizing members of the plurality of stabilizing members. Each group of stabilizing members may comprise more than one stabilizing member. The plurality of stabilizing members may be coupled to, or connected to, the distal lobe. Each of the plurality of stabilizing members may include a backing portion. Each backing portion may be U-shaped. Each backing portion may comprise one or more eyelets. A pair of eyelets may be provided for the respective pair of engagement features of each backing portion. The eyelets of the plurality of stabilizing members may be coupled to, or connected to, the distal lobe. Each of the plurality of stabilizing members may include a backing portion, the backing portion including two legs extending therefrom, each of the two legs extending to a terminal end, the terminal end of each leg forming one of the pair of engagement features of the first group of engagement features, each of the pair of engagements features of the second group of engagement features extending from a respective one of the two legs. Each of the plurality of stabilizing members may include a single engagement feature of the first group of engagement features, and a single engagement feature of the second group of engagement features. Each of the plurality of stabilizing members may include a backing portion, the backing portion including two legs extending therefrom, a first one of the two legs terminating in the single engagement feature of the first group of engagement features, and a second one of the two legs terminating in the single engagement feature of the second group of engagement features, the first one of the two legs having a length that is longer than a length of the second one of the two legs. Each of the plurality of stabilizing members may include a pair of engagement features of the first group of engagement features, and a single engagement feature of the second group of engagement features. Each of the plurality of stabilizing members may include a backing portion, the backing portion including two legs extending therefrom, each of the two legs extending to a terminal end, the terminal end of each leg forming one of the pair of engagement features of the first group of engagement features, the single engagement feature of the second group of engagement features being formed integrally with the backing portion. Each of the plurality of stabilizing members may include a backing portion, the backing portion including two legs extending therefrom, each of the two legs extending to a terminal end, the terminal end of each leg forming one of the pair of engagement features of the first group of engagement features, the single engagement feature of the second group of engagement features being coupled, via a connector, to the backing portion.

According to a further aspect of the disclosure, a medical device for occluding a left atrial appendage (“LAA”) includes a proximal disk configured to cover an ostium of the LAA in an implanted condition of the medical device, a distal lobe configured to be received within a cavity of the LAA in the implanted condition of the medical device, and a connecting member connecting the proximal disk to the distal lobe. The medical device includes a first group of stabilizing members, each stabilizing member in the first group having a first engagement feature configured to engage tissue surrounding the LAA in the implanted condition of the medical device. The medical device includes a second group of stabilizing members, each stabilizing member in the second group having a second engagement feature configured to engage tissue surrounding the LAA in the implanted condition of the medical device. The medical device includes a third group of stabilizing members, each stabilizing member in the third group having a third engagement feature configured to engage tissue surrounding the LAA in the implanted condition of the medical device. In an unbiased condition of the medical device, the distal lobe has an axial length extending between a proximal face and a distal face of the distal lobe, the first engagement features are positioned at a first distance along the axial length, the second engagement features are positioned at a second distance along the axial length, and the third engagement features are positioned at a third distance along the axial length, the first distance being different than the second distance, the second distance being different than the third distance, the third distance being different than the first distance. The axial length may be about 10 mm. The first distance may be between about 8 mm and about 10 mm, such that the first engagement features are positioned between about 0 mm and about 2 mm proximally of the distal face of the distal lobe. The second distance may be between about 3.3 mm and about 4.3 mm. The third distance may be between about 4.4 mm and about 5.4 mm. The medical device may include a total number of stabilizing members in the first group that is at least twice as many as a total number of stabilizing members in the second group and that is at least twice as many as a total number of stabilizing members in the third group. A single stabilizing member in either the second group or the third group may be positioned between each circumferentially adjacent pair of stabilizing members in the first group. Each stabilizing member may be separate from the other stabilizing members, and each group of stabilizing members may comprise more than one stabilizing member, and each stabilizing member may be connected to the distal lobe. Each stabilizing member in the first, second, and third groups may be formed of a wire. Each stabilizing member in the first, second, and third groups may be cut from a sheet of material. Each first engagement feature, each second engagement feature, and each third engagement feature may be a hook.

According to a further aspect of the disclosure there is provided a medical device for occluding a left atrial appendage (“LAA”), the medical device comprising: a proximal disk configured to cover an ostium of the LAA in an implanted condition of the medical device; a distal lobe configured to be received within a cavity of the LAA in the implanted condition of the medical device; a connecting member connecting the proximal disk to the distal lobe; and a plurality of stabilizing members, the plurality of stabilizing members including a first group of engagement features configured to engage tissue surrounding the LAA in the implanted condition of the medical device and a second group of engagement features configured to engage tissue surrounding the LAA in the implanted condition of the medical device, wherein in an unbiased condition of the medical device, the distal lobe has an axial length extending between a proximal face and a distal face of the distal lobe, the first group of engagement features of the plurality of stabilizing members being positioned distal to the second group of engagement features of the plurality of stabilizing members. The second group of engagement features of the plurality of stabilizing members may be positioned at least 33% of the axial length beyond the proximal face of the distal lobe. The plurality of stabilizing members may comprise a first group of stabilizing members and a second group of stabilizing members. Each stabilizing member in the first group of stabilizing members may have an engagement feature to provide the first group of engagement features. Each stabilizing member in the second group of stabilizing members may have an engagement feature to provide the second group of engagement features. The plurality of stabilizing members may comprise a third group of stabilizing members. Each stabilizing member in the third group may have a third engagement feature configured to engage tissue surrounding the LAA in the implanted condition of the medical device. The first engagement features may be positioned at a first distance along the axial length, the second engagement features may be positioned at a second distance along the axial length, and the third engagement features may be positioned at a third distance along the axial length. The first distance may be different than the second distance, the second distance may be different than the third distance, and the third distance may be different than the first distance.

According to a further aspect of the disclosure there is provided a medical device comprising a lobe and a plurality of stabilizing members that are connected to the lobe, the plurality of stabilizing members including a first group of engagement features configured to engage tissue surrounding the lobe in an implanted condition of the medical device and a second group of engagement features configured to engage tissue surrounding the lobe in the implanted condition of the medical device, wherein, in an unbiased condition of the medical device, the lobe has an axial length extending between a proximal face and a distal face of the distal lobe, and wherein the first group of engagement features of the plurality of stabilizing members are positioned distal to the second group of engagement features of the plurality of stabilizing members.

It will be appreciated that, in general, a feature described in relation to one embodiment may be provided in combination with another embodiment.

### BRIEF DESCRIPTION OF THE DRAWINGS

FIG. 1 illustrates a known medical device.

FIGS. 2A-2C are a schematic diagram of the known medical device shown in FIG. 1 under radial compression.

FIG. 3 is a schematic diagram of a delivery system in accordance with the present disclosure.

FIG. 4 is a highly schematic view of the medical device of FIG. 1 implanted within an exemplary LAA.

FIGS. 5-6 show an example of an occluder according to an aspect of the disclosure, the occluder being in an expanded condition.

FIGS. 7A-B are exemplary embodiments of a stabilizing member with at least one eyelet formed by wire or by laser cut design in accordance with the present disclosure.

FIG. 8 is an enlarged view of a stabilizing member connected to an occluder.

FIG. 9A is another view of an occluder similar to that shown in FIGS. 5A-B, with FIG. 9B showing a pattern for the smaller stabilizing member before being shaped or contoured.

FIG. 10A is another example of an occluder with two rows of engagement features of stabilizing members at different axial positions, with FIGS. 10B-10D showing examples of patterns for the stabilizing member before being shaped or contoured.

FIG. 11 is another example of an occluder with two rows of engagement features of stabilizing members at different axial positions.

FIG. 12A is another example of an occluder with two rows of engagement features of stabilizing members at different axial positions, with FIGS. 12B-12C showing examples of the stabilizing members.

FIG. 13 is another example of an occluder with two rows of engagement features of stabilizing members at different axial positions, including two rows of smaller stabilizing members similar to the smaller stabilizing members shown in FIGS. 5-6.

FIGS. 14-15 are top perspective and bottoms views, respectively, of an occluder according to another aspect of the disclosure.

FIG. 16 is a side view of the occluder of FIGS. 14-15.

FIG. 17 is a simplified side view of the occluder of FIGS. 14-16.

### DETAILED DESCRIPTION OF THE DISCLOSURE

The present disclosure relates generally to medical devices that are used in the human body. Specifically, the present disclosure provides medical devices including occlusion devices having stabilizing members for enhancing engagement of the occluder with the tissue in which it is implanted. The disclosed embodiments may lead to more consistent and improved patient outcomes. It is contemplated, however, that the described features and methods of the present disclosure as described herein may be incorporated into any number of systems as would be appreciated by one of ordinary skill in the art based on the disclosure herein.

Although the exemplary embodiment of the medical device is described as treating a target site including a LAA, it is understood that the use of the term “target site” is not meant to be limiting, as the medical device may be configured to treat any target site, such as an abnormality, a vessel, an organ, an opening, a chamber, a channel, a hole, a cavity, or the like, located anywhere in the body. The term “vascular abnormality,” as used herein is not meant to be limiting, as the medical device may be configured to bridge or otherwise support a variety of vascular abnormalities. For example, the vascular abnormality could be any abnormality that affects the shape of the native lumen, such as an atrial septal defect, a lesion, a vessel dissection, or a tumor. Embodiments of the medical device may be useful, for example, for occluding a patent foramen ovalis (“PFO”), atrial septal defect (“ASD”), ventricular septal defect (“VSD”), or patent ductus arteriosus (“PDA”), as noted above. Furthermore, the term “lumen” is also not meant to be limiting, as the vascular abnormality may reside in a variety of locations within the vasculature, such as a vessel, an artery, a vein, a passageway, an organ, a cavity, or the like. As used herein, the term “proximal” refers to a part of the medical device or the delivery device that is closest to the operator, and the term “distal” refers to a part of the medical device or the delivery device that is farther from the operator at any given time as the medical device is being delivered through the delivery device. In addition, the terms “deployed” and “implanted” may be used interchangeably herein.

Some embodiments of the present disclosure provide an improved percutaneous catheter directed intravascular occlusion device for use in the vasculature in patients' bodies, such as blood vessels, channels, lumens, a hole through tissue, cavities, and the like, such as a LAA. Other physiologic conditions in the body occur where it is also desirous to occlude a vessel or other passageway to prevent blood flow into or therethrough. These device embodiments may be used anywhere in the vasculature where the anatomical conditions are appropriate for the design.

The medical device may include one or more layers of occlusive material, wherein each layer may be comprised of any material that is configured to substantially preclude or occlude the flow of blood so as to facilitate thrombosis. As used herein, “substantially preclude or occlude flow” shall mean, functionally, that blood flow may occur for a short time, but that the body's clotting mechanism or protein or other body deposits on the occlusive material results in occlusion or flow stoppage after this initial time period.

Some embodiments of the present disclosure may be formed by a plurality of wire strands having a predetermined relative orientation with respect to one another. However, it is understood that according to additional embodiments of the present disclosure, that the medical device could be etched or laser cut from a tube, or the device could comprise an occlusion material coupled to a scaffolding structure or a plurality of slices of a tubular member coupled together.

The present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

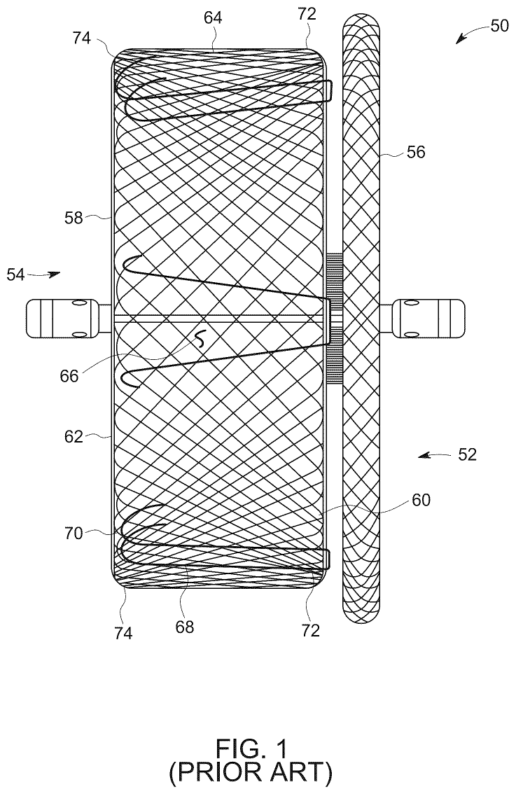

In at least some conventional or known medical devices used for the occlusion of abnormalities, such as a medical device 50 shown in FIG. 1, medical device 50 includes a proximal end 52 and a distal end 54, with a disk 56 at proximal end 52 and a lobe 58 at distal end 54. The lobe 58 has a proximal edge 60 (also referred to as a proximal face), a distal edge 62 (also referred to as a distal face), and a middle or central portion 64 that define a cavity 66. The medical device 50 also includes stabilizing members (which may be formed using wire(s)) 68 secured to a radially outer or circumferential surface of middle portion 64. The stabilizing members 68 terminate in an engagement feature (which may be a hook or a hooked end of a wire forming the stabilizing member) 70 at free ends thereof, and thereby facilitate retention of the medical device 50 at a target site and preventing the medical device 50 from becoming dislodged from the target site after deployment.

In this known medical device 50, proximal edge 60 and distal edge 62 adjoin middle portion 64 at a first relatively blunt or sharp (e.g., non-rounded) transition 72 and a second blunt transition 74, respectively. First blunt transition 72 connects proximal edge 60 to middle portion 64 by an approximately 90 degree angle. Likewise, second blunt transition 74 connects distal edge 62 to middle portion 64 by an approximately 90 degree angle. First blunt transition 72 and second blunt transition 74 partially define a generally rectangular cross section to lobe 58, leading to relatively blunt circumferential edges of the device and relatively high radial force applied to the surrounding tissue.

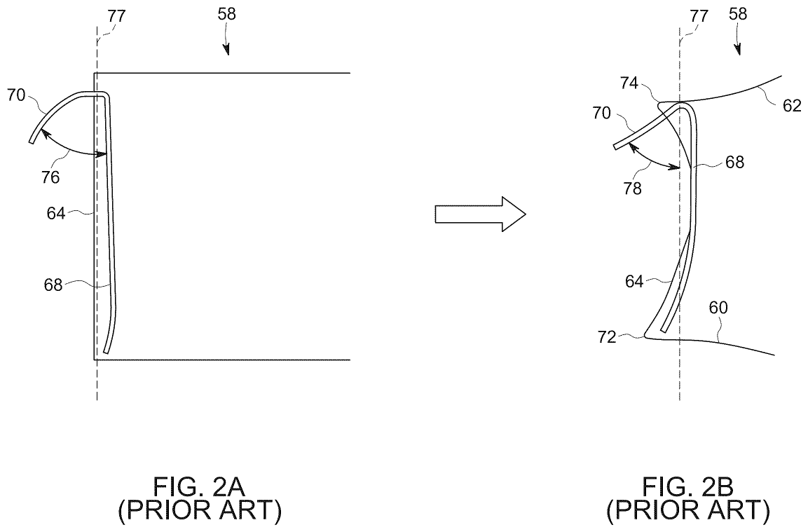

Turning now to FIGS. 2A-2C, medical device 50 before and after undergoing radial compression is depicted. Before radial compression (FIG. 2A) is applied to lobe 58, the outer surface of middle portion 64 is linear or extends generally perpendicular to proximal and distal faces 60, 62. Each engagement feature (e.g. hook) 70 of a corresponding stabilizing member (e.g. stabilizing wire) 68 is at a first angle 76 with respect to a generally longitudinal direction 77. When radial compression is applied to lobe 58 (FIGS. 2B and 2C), proximal and distal faces 60, 62 flex and bow outwardly (e.g., axially outward), and middle portion 64 of lobe 58 flexes and bows inwardly, in response to the applied force. The approximately 90 degree angle of first blunt transition 72 and second blunt transition 74 force the outer surface of middle portion 64 to transition from linear to concave when proximal and distal faces 60, 62 bow outwardly. The concave shape adopted by lobe 58 also shifts the position of stabilizing members 68, such that stabilizing members 68 at least partially contract and engagement features 70 transition from first angle 76 to a second, greater angle 78. At second angle 78, engagement features 70 are oriented more directly towards the adjacent tissue, than when engagement features 70 are at first angle 76. This shift in orientation of stabilizing members 68, and therefore engagement members 70, can lead to an increase in interactions between engagement members 70 and the adjacent tissue at the target site within the patient's body. The increased interaction with tissue can lead to adverse side effects such as late pericardial effusion.

The medical devices of the present disclosure may include a rounded lobe, for example as described in greater detail in U.S. Patent Application Publication No. 2022/0008050, the disclosure of which is hereby incorporated by reference herein. The use of a more rounded lobe may lead to a more uniform radial compression, reduction in radial force applied to surrounding tissue, and reduction in variability of the hook angle of the stabilizing wires, minimizing potential disadvantages of known medical devices.

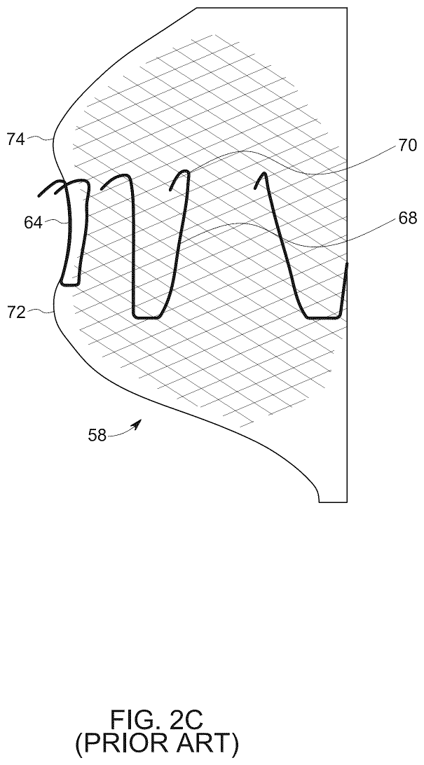

Turning now to FIG. 3, a schematic diagram of a delivery system 100 is shown. Delivery system 100 includes a delivery device 102 including a catheter 104 and a coupling member 106 configured to couple a distal end of a delivery cable 108 to a medical device 110 (which may be any of the occluders described herein) for facilitating the deployment of medical device 110 at a target site. Medical device 110 is deployed to treat the target site, and, in the example embodiment, is an occlusion device (“occluder”).

Due to the wide variety of LAA anatomy and possible implant locations for any given implant size, it may be useful to include additional or alternatively-configured stabilizing members (e.g. stabilizing wires) or engagement features (e.g. hooks or hooked ends of a wire) beyond those shown in the embodiments of FIGS. 1-2C to help ensure stability of the occluder in most configurations of anatomy and implant size.

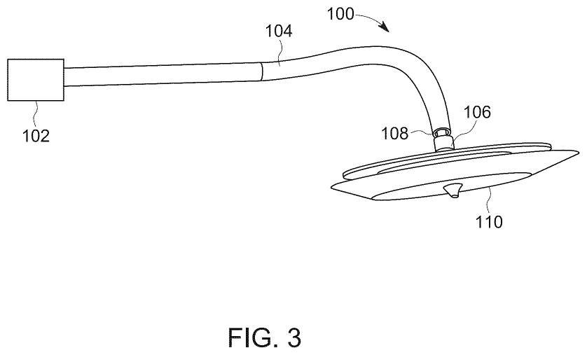

FIG. 4 is a highly schematic illustration of the medical device 50 used as an occluder that has been deployed within a LAA of a patient. Nearby anatomy, including exemplary locations of the pulmonary vein PV, circumflex artery CX, and mitral valve MV are shown for additional context. In the example configuration of FIG. 4, the occluder 50 has been implanted in a manner that the top of the occluder 50 (in the orientation of the view of FIG. 4) is in contact with most of the tissue on the top side of the device, as indicated at location 1. However, on the bottom side of the occluder (in the orientation of the view of FIG. 4), only the proximal portion of the lobe 58 is engaged with the tissue. This configuration may result in a scenario in which the engagement features (e.g. hooks) of stabilizing members (e.g. stabilizing wires) 68 of the occluder 50 are not engaged with tissue in that portion of the anatomy, as indicated at location 2.

If the type of implantation shown in FIG. 4 occurs, the occluder 50 may be unstable following implantation or deployment, particularly on the bottom side of the occluder 50 (in the orientation of the view of FIG. 4), for example at or near location 2. During implantation of the occluder 50, after the occluder 50 has expanded into the LAA but prior to the delivery device 102 being detached from the occluder 50 (e.g. prior to uncoupling the coupling member 106 from the occluder 50), a traction test or tension test (which may also be referred to as a “tug test”) may be performed. Generally, a traction test (or tension test or tug test) includes pulling proximally on the delivery device 102 to place a slight proximal tension force on the occluder 50 (e.g. via the delivery cable 108). This slight proximal tension force or “tug” may cause the stabilizing members 68 (and/or engagement features or hooks) to gain better engagement with the surrounding tissue and confirm that the occluder 50 is securely positioned within the LAA (e.g. by confirming that the tension or “tug” does not cause a change in position or orientation of the occluder 50 relative to the LAA despite the applied force). However, if the occluder 50 has the configuration shown in FIG. 4 (or a similar configuration), when the tension or tug test is performed, or if the occluder 50 is left in place under compression and pressure, the occluder can tilt and migrate until the non-engaged stabilizing wires 68 (and/or non-engaged engagement features or hooks) (e.g. at or around location 2) engage tissue. This scenario can lead to gaps around the sealing surface of the occluder 50, or even embolization of the occluder 50.

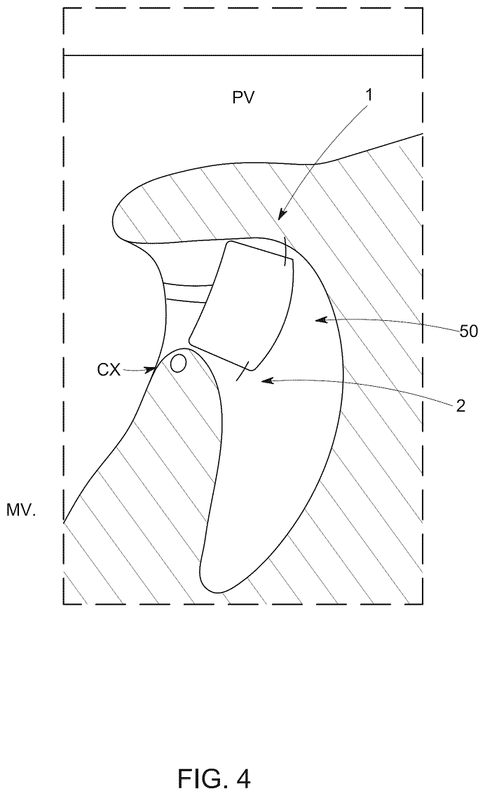

FIG. 5 shows an example of an occluder 200 according to an aspect of the disclosure, the occluder 200 being in an expanded condition. In some examples, the occluder 200 is formed of braided mesh (e.g. strands of shape-memory material braided together) or laser cut from a tube of shape-memory material, and may include a proximal disk 210, a distal lobe 230, and a connecting portion 220 connecting the two. Distal lobe 230, as well as all other distal lobes described herein, may have a generally cylindrical shape or configuration, although one or both ends of the distal lobe 230 may be curved (to varying degrees) and the proximal and distal faces of the distal lobe may not have flat (or at least perfectly flat) shapes. Proximal disk 210, as well as all other proximal disks described herein, may have a generally flat disk shape or configuration. The distal lobe 230 may be intended for placement within the LAA while the proximal disk 210 may be intended to cover the ostium of the LAA. Compared to occluder 50, which has stabilizing members 68 with engagement features (e.g. hooks) positioned so that all of the hooks are positioned near the distal face 62, occluder 200 may include at least a first group of stabilizing members (which may be formed from wire(s)) 240 and a second group of stabilizing members (which may be formed from wire(s)) 250 having a different configuration than the first group. For example, stabilizing members in the first group 240 may be generally similar in size and position as stabilizing members 68, with engagement features (e.g. hooks or hooked ends of a wire), in particular the distal ends of the engagement features, being positioned near the distal face of the distal lobe 230. However, stabilizing members in the second group 250 may be smaller and/or shorter, so that the engagement features (e.g. hooks or hooked ends of a wire), in particular the distal ends of the engagement features, are positioned near a mid-length segment (in the axial direction) of the distal lobe 230. In other words, the engagement features (e.g. hooks) of stabilizing members 250 may be positioned significantly proximal relative to the engagement features (e.g. hooks) of stabilizing members 240. It should be understood that, in FIG. 5, one stabilizing member 250 is labeled and one stabilizing member 240 is labeled, but a plurality of each type of stabilizing member 240, 250 may be provided, for example in an alternating configuration around the circumference of the distal lobe 230. Compared to the stabilizing members 68 of occluder 50, the configuration of the groups of stabilizing members 240, 250 of occluder 200 may increase the resilience of the occluder to embolization or motion in an implanted condition similar to that shown in FIG. 4, at least partially due to the second group of stabilizing members 250 being added in a second row, more proximal on the lobe 230 compared to the first group of stabilizing members 240.

The location of the engagement features (e.g. hooks or distal hooked ends of wire(s)) of the stabilizing members in the second group 250 may be selected to account for different countervailing objectives. For example, if the engagement features of the stabilizing members in the second group 250 are positioned too far distally, the problem produced in an implanted condition similar to that shown in FIG. 4 may not be solved. However, if the engagement features of the stabilizing members in the second group 250 are positioned too far proximally, they may pose risk to surrounding anatomy such as the circumflex artery CX shown in FIG. 4.

For devices similar to occluder 50 (and/or occluder 200), instructions for use may typically indicate that the occluder 50 (or occluder 200) should be implanted so that at least two-thirds of the length of the lobe 58 (or lobe 230) is positioned beyond (e.g., distally to) the circumflex artery CX. In such examples, the proximal third of the lobe 58 (or lobe 230) has the potential to overlap with the circumflex artery CX. To avoid interaction with the circumflex artery CX, in one design, engagement features (e.g. hooks or hooked ends of wire(s)) of the stabilizing members in the second group 250 may be positioned a minimum of the proximal third of the length of the lobe 58 (or lobe 230). In other words, if the axial length of the lobe 58 (or lobe 230) from the proximal edge to the distal edge is a unit of length, the engagement features of the stabilizing members of the second group 250 may be positioned a minimum of one-third of that unit of length beyond the proximal edge of the lobe 58 (or lobe 230) (that is, the stabilizing members in the second group 250 may be positioned at least 33% of the axial length beyond the proximal face of the distal lobe 230) to prevent potential damage to the circumflex artery CX if the engagement features partially penetrate into the tissue wall of the heart. In some examples, the lengths described in this paragraph refer to dimensions when the occluder 50 (or occluder 200) is in an unbiased condition, for example in the absence of applied forces. Further, when referring to the stabilizing members 250 being positioned at least 33% of the axial length beyond the proximal face of the distal lobe 230, the structure of the stabilizing member 250 having such positioning is preferably the tip (e.g. the terminal end configured to actually frictionally engage tissue), although in other embodiments the structure of the stabilizing member 250 having such positioning may be the distal-most end of the stabilizing member 250.

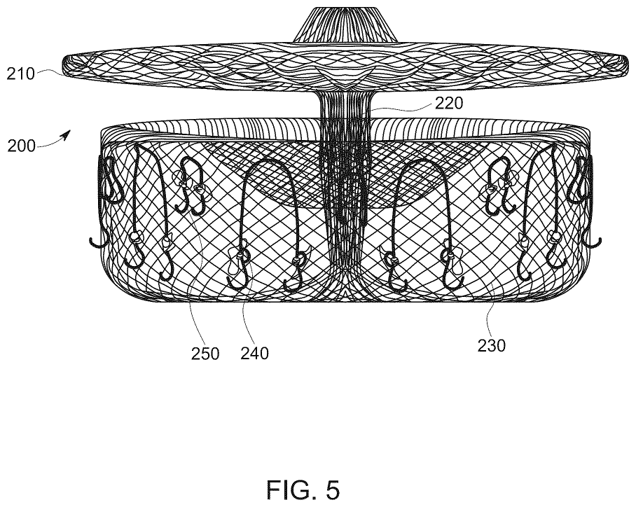

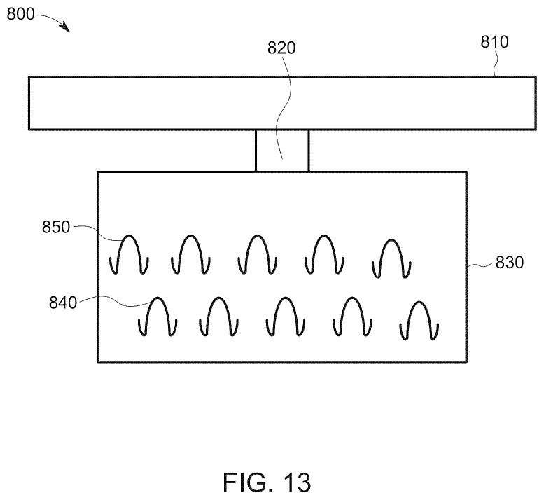

For example, FIG. 6 is another view of occluder 200 similar to FIG. 5, showing a dimension A extending from the proximal face of the lobe 230 toward the distal face. The dimension A may represent the distance between the proximal face of the lobe 230 and the position of the tips or terminal ends of the engagement features (e.g. hooks or hooked ends of wire(s)) of the stabilizing members in the second group 250. As noted above, the dimension A preferably has a length of at least one-third the total length of the lobe 230, the total length being represented by the dimension B in FIG. 6. In some examples, occluders 200 may be provided in various sizes, which may include different sized lobes. For example, a small size occluder 200 may include a lobe having a length B of about 7.5 mm, while a large size occluder 200 may include a lobe having a length B of about 10 mm. In some examples, for the small size occluder 200, the dimension A may be about 3.5 mm±0.5 mm, while for the large size occluder 200, the dimension A may be about 4.5 mm±0.75 mm. In some examples, the dimension A may be between about 33% and about 55% of the dimension B, including for example about 35%, about 40%, about 45%, and about 50%. In other configurations, including for example those shown in FIGS. 14-17 and described in greater detail below, the proximal dimension A can vary slightly per pair of stabilizing members, which may create a lower required force when recapturing the device because the engagement features (e.g. hooks or hooked ends of wire(s)) do not all contact the delivery sheath at the same time, as they are offset at least slightly.

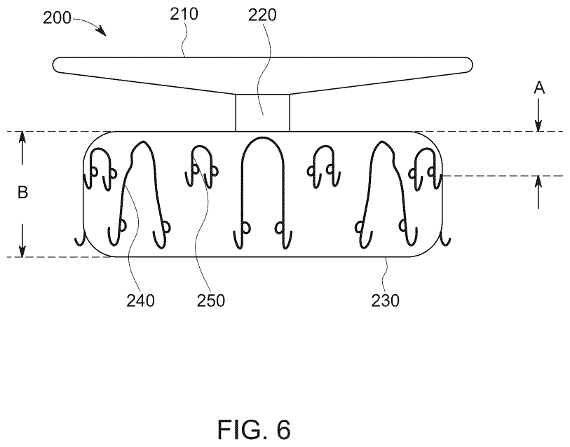

It should be understood that the stabilizing members and/or the engagement features (e.g. hooks) thereof described herein may take any suitable form. Two examples of a stabilizing members 300 are shown in FIGS. 7A-B. Additional detail regarding the specific structure of stabilizing member 300, and other suitable options for stabilizing members, is described in greater detail in U.S. Patent Application Publication No. 2022/0280166, the disclosure of which is hereby incorporated by reference herein. In the examples of FIGS. 7A-B, stabilizing member 300 may be formed from wire or other material described herein, or formed from a laser cut design as described herein, and may include at least one eyelet 307. The stabilizing member 300 may generally include a backing portion 304, which may include a general “U”-shape design positioned opposite the engagement features, with two legs extending from the backing portion 304 to the opposite engagement features 302, which may be a hook, a hooked end, or tip configured to engage tissue. The eyelet(s) 307, if included, may be used to couple (e.g., via suturing) the stabilizing member 300 to the occluder (e.g., occluder 50 or 200). As should be understood, features like the length of the stabilizing member 300 between the backing portion 304 and the engagement features 302 may be adjusted as desired to achieve the desired effect (e.g., a shorter length for use in the group of stabilizing members 250 of FIGS. 5-6, or a longer length for use in the group of stabilizing members 240 of FIGS. 5-6). In some examples, particularly for longer lobes, the different groups of stabilizing members may have equal lengths.

One factor for consideration described above is the axial position of the engagement features of the one or more groups of stabilizing members (e.g., groups 240, 250) relative to the length of lobe 230 and relative to the eventual implanted position within the LAA. Another factor in the design is the radial spacing of the stabilizing members. For example, in the embodiment shown in FIGS. 5-6, each stabilizing member in the second group 250 is positioned between a pair of stabilizing members in the first group 240, with the circumferential spacing between each adjacent stabilizing member being substantially even. In other words, in the embodiment of FIGS. 5-6, the stabilizing members are positioned to create substantially even circumferential spacing between the stabilizing members so that occluder 200 has no or low sensitivity to rotational orientation within the LAA. Typical delivery systems for LAA occlusion devices do not include the capability to control the rotation of the occluder (the rotation being defined about a longitudinal axis passing through the center of the connection portion 220 in a proximal-to-distal direction, for example). Additionally, there is large variety of LAA shapes among patients, and many LAA anatomies are elliptical or non-symmetric. Thus, it may actually be preferable to not require any rotational control of the occluder 200, and as such, it may be important for the occluder 200 to be rotationally symmetric about one or more planes to reduce or eliminate the impact of implantation in one rotational position compared to another rotational position. In some examples, the angle between each adjacent pair of stabilizing members in the first group 240, and/or between each adjacent pair of stabilizing members in the second group 250, is between about 20 degrees and about 65 degrees. However, it should be understood that for larger size versions of the occluder 200 (which may have more total stabilizing members), the angle may be smaller, and for smaller size versions of the occluder 200 (which may have fewer total stabilizing members), the angle may be larger.

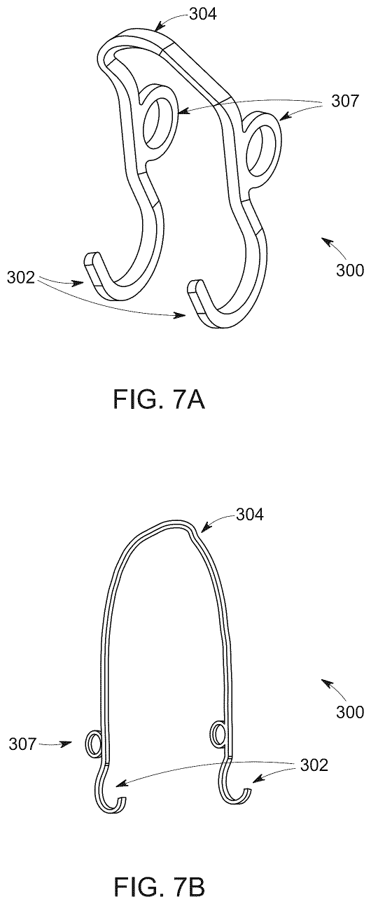

FIG. 8 is an enlarged view of a stabilizing member 400 (e.g. stabilizing wire) coupled to a lobe of an occluder. In this particular example, stabilizing member 400 has the general design shown in FIGS. 7A-B and is a smaller stabilizing member of the group of stabilizing members 250 of FIG. 5. In the view of FIG. 8, the top of the figure is the proximal face of the lobe 230, with the distal face of the lobe not being shown but being positioned in the direction of the bottom of the figure. As can be seen, in this example, the backing portion 404 of the stabilizing member 400 is positioned radially outward of the braided mesh that forms the lobe 230, with the two legs extending from the backing portion 404 being largely positioned radially inside the lobe 230. A connector 410, which in this case is a suture or other string-like member, connects, tics, or otherwise couples each eyelet 407 to a portion of the braid that forms the lobe 230. The engagement features 402 (e.g. hooks, hooked ends, or tips) of the stabilizing members 400 extend through an opening in the braided mesh forming the lobe 230 so that the engagement features 402 extend from the radially inner portion of the lobe 230 radially outward of the lobe 230 so that the terminal ends are available for engagement with tissue upon implantation. In some examples, although stabilizing member 400 represents the shorter stabilizing member of the second group 250 of FIGS. 5-6, the stabilizing members used for the first group 240 of FIGS. 5-6 may be similar or identical from the eyelets 407 to the engagement features 402, with the only significant difference being the length of the legs between the backing portion 404 and the eyelets 407.

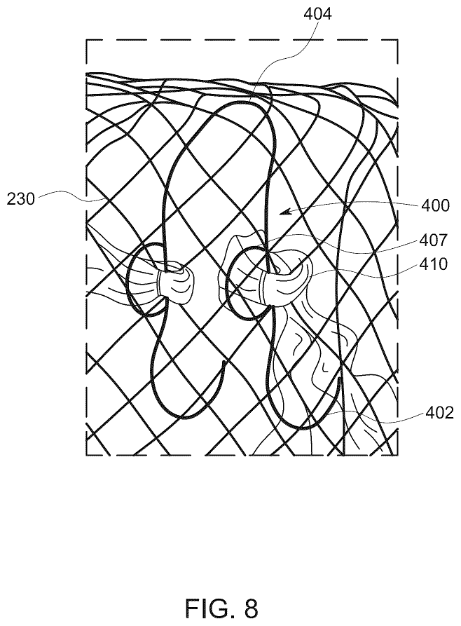

FIG. 9A is another view of an occluder 200 which may be similar or identical to that shown in FIGS. 5A-B, which may include a proximal disk 210 connected to a distal lobe 230 by a connecting portion 220. In this particular example, the smaller stabilizing members 250 within the second group may be formed by laser cutting the stabilizing members 250 from a sheet of material (e.g., nitinol) and then setting the stabilizing member 250 into the desired shape (e.g., similar to that shown in FIG. 7A). FIG. 9B shows an example of the cut pattern for the smaller stabilizing member 250 before being shaped or contoured. For example, the stabilizing member of FIG. 9B can be laser cut and then shaped to the desired shape, or in other examples formed from a wire and then shaped to the desired shape. The view of FIG. 9B illustrates examples of positions of the backing portion 254, two eyelets 257, and two engagement features (e.g. hooks or hooked ends) 252, prior to being shaped (or shape-set) into the corresponding features shown in, for example, FIG. 7A.

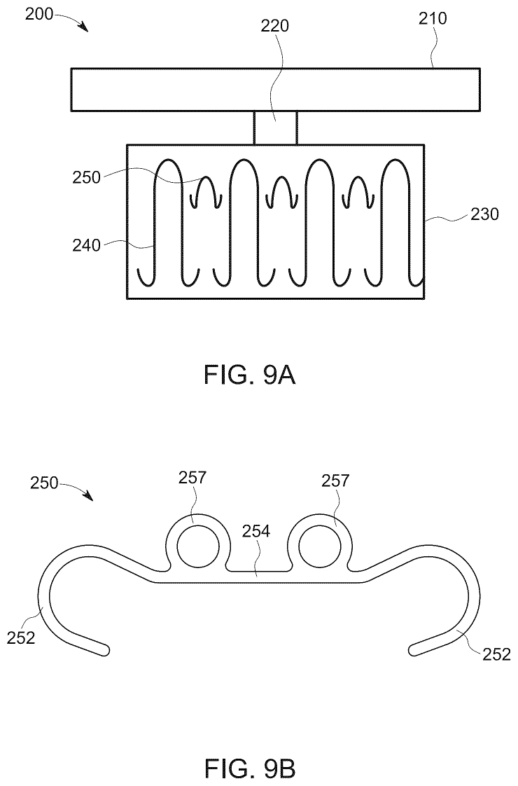

FIG. 10A is another example of an occluder with two rows of engagement features of stabilizing members at different axial positions. For example, instead of having two groups of different stabilizing members with engagement features (e.g. hooks or hooked ends) at different positions, the example of FIG. 10A includes stabilizing members that are all similar or identical to each other, but that each include one or more engagement features closer to the distal end of the lobe, as well as one or more additional engagement features nearer the mid-length segment of the distal lobe. In this example, occluder 500 may be substantially similar or identical to occluder 200 with the exception of the stabilizing members. For example, occluder 500 may include a proximal disk 510 connected to a distal lobe 530 by a connection portion 520. In some examples, each stabilizing member 550 may include a backing portion 554 and two distal engagement features (e.g. distal hooks) 552 configured to engage tissue. A pair of proximal engagement features (e.g. proximal hooks) 556 may be positioned on the legs extending between the backing portion 554 and the distal engagement features 552. As should be understood, the relationship between the position of the distal engagement features 552 and the proximal engagement features 556 may be similar or identical to the engagement features of the stabilizing members 240 in the first group of occluder 200 and the engagement features of the stabilizing members 250 in the second group of occluder 200.

FIGS. 10B-10D show examples of patterns for the stabilizing member before being shaped or contoured, which show examples of how the different engagement features (e.g. hooked ends) may be positioned relative to each other before being contoured. For example, FIG. 10B shows one example of a cut pattern 550a that may be used, for example by laser-cutting a flat sheet of material (e.g., nitinol), to create a structure that may then be used to form stabilizing member 550, for example by shape-setting the cut structure. As shown in FIG. 10B, the cut pattern 550a may include a portion 554a that may be contoured into a backing portion, a pair of proximal engagement features (e.g. proximal hooks) 556a, a pair of distal engagement features (e.g. distal hooks) 552a, and a pair of eyelets 557a each positioned between one of the proximal engagement features (e.g. proximal hooks) 556a and one of the distal engagement features (e.g. distal hooks) 552a on the same leg of the stabilizing member.

FIG. 10C shows another example of a cut pattern 550b that may be used, for example by laser-cutting a flat sheet of material (e.g., nitinol), to create a structure that may then be used to form stabilizing member 550, for example by shape-setting the cut structure. As shown in FIG. 10C, the cut pattern 550b may include a portion 554b that may be contoured into a backing portion, a pair of proximal engagement features (e.g. proximal hooks) 556b, a pair of distal engagement features (e.g. distal hooks 552b), and a pair of eyelets 557b each positioned between one of the proximal engagement features 556b and one of the distal engagement features 552b on the same leg of the stabilizing member. Cut pattern 550b is generally similar to cut pattern 550a, with two main differences. First, while each eyelet 557a of cut pattern 550a is formed entirely on one side of the leg extending between the backing portion 554a and the distal engagement feature 552a, each eyelet 557b of cut pattern 550b provides a transition of the leg extending between the backing portion 554b and the distal engagement feature 552b. Stated otherwise, each leg extending from the backing portion 554b meets an eyelet 557b on a rear portion of the eyelet 557b, with the leg continuing to extend from the front portion of the eyelet 557b to form the distal engagement feature 552b. With this configuration, the distal engagement features 552b may be positioned generally aligned with the proximal engagement features 556b, despite the second main difference, described below. Cut pattern 550b may include a more complete proximal engagement feature (e.g. proximal hook) 556b compared to proximal engagement feature (e.g. proximal hook) 556a. For example, whereas the hooked portion of proximal engagement feature 556a extends directly from the leg extending between the backing portion 554a and the distal engagement feature 552a, the hooked portion of proximal engagement feature 556b may include an extension so that the shape of the proximal engagement feature 556b substantially matches the shape of distal engagement feature 552b. This may provide increased uniformity between the proximal engagement features 556b and the distal engagement features 552b.

FIG. 10D shows another example of a cut pattern 550c that may be used, for example by laser-cutting a flat sheet of material (e.g., nitinol), to create a structure that may then be used to form stabilizing member 550, for example by shape-setting the cut structure. Cut pattern 550c is identical to cut pattern 550b in most respects, including in that it includes a backing portion 554c, proximal engagement features (e.g. proximal hooks) 556c and distal engagement features (e.g. distal hooks) 552c, and eyelets 557c. While each of these individual components of cut pattern 550c may have substantially identical shapes to their counterparts in cut pattern 550b, the main difference between cut pattern 550c and cut pattern 550b is the position of the proximal engagement feature 556c relative to the adjacent portion of the leg extending between the backing portion 554c and eyelet 557c. For example, in cut pattern 550c, the proximal engagement feature 556c has more spacing from the adjacent leg compared to the spacing between proximal engagement feature 556b and its adjacent leg. This may be achieved, in one example, by including a more acute angle where the leg emerges from the backing portion 554c, compared to the smoother transition between backing portion 554b and the adjacent leg in cut pattern 550b.

Regardless of whether cut pattern 550a, 550b, 550c, or another cut pattern is used to form stabilizing members 550, the resulting configuration may help ensure that there are an equal number of distal engagement features (e.g. distal hooks) 552 as proximal engagement features (e.g. proximal hooks) 556, and also help ensure consistent spacing between the distal engagement features 552 and proximal engagement features 556. In embodiments that use cut pattern 550b or 550c, or similar cut patterns, the additional clearance provided adjacent the proximal engagement features 556b, 556c may facilitate relatively independent unfurling of the proximal engagement features 556 during recapture of the occluder 500 within a delivery device. The use of stabilizing members 550 may also reduce complexity, at least in that separate types of stabilizing members (e.g. one group of large stabilizing members and one group of small stabilizing members) may be avoided.

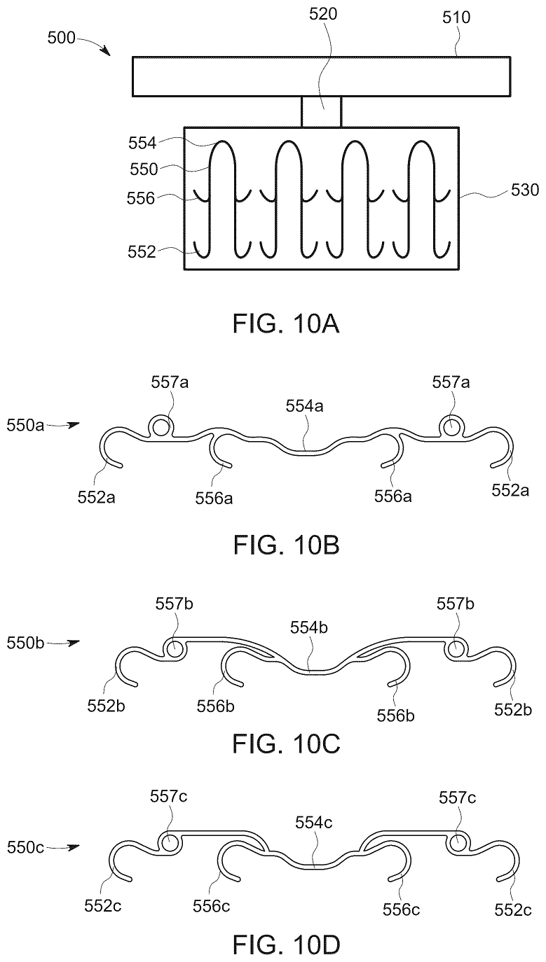

FIG. 11 is another example of an occluder with two rows of engagement features of stabilizing members at different axial positions. In this example, each stabilizing member has a pair of engagement features (e.g. hooks or hooked ends), but one of the two engagement features has a relatively distal position while the other of the two engagement features has a relatively proximal position. In this example, occluder 600 may be substantially similar or identical to occluder 200 with the exception of the stabilizing members. For example, occluder 600 may include a proximal disk 610 connected to a distal lobe 630 by a connection portion 620. In some examples, each stabilizing member 650 may include a backing portion 654, one distal engagement feature (e.g. distal hook) 652 configured to engage tissue, and one proximal engagement feature (e.g. proximal hook) 656. As should be understood, the relationship between the position of the distal engagement feature 652 and the proximal engagement feature 656 may be similar or identical to the engagement features of the stabilizing members 240 in the first group of occluder 200 and the engagement features of the stabilizing members 250 in the second group of occluder 200. Whereas other stabilizing members described above are generally symmetric about a centerline, stabilizing members 650 are not. For example, stabilizing members 650 may have an offset configuration in which one relatively short leg extends from backing portion 654 to proximal engagement features 656, and one relatively long leg extends from backing portion 654 to distal engagement feature 652. In this example, the use of stabilizing members 650 may help ensure that there are an equal number of distal engagement features 652 as proximal engagement features 656, and also help ensure consistent spacing between the distal engagement features 652 and proximal engagement features 656. Further, the use of stabilizing members 650 may also reduce complexity, at least in that separate types of stabilizing members (e.g. one group of large stabilizing members and one group of small stabilizing members) may be avoided. Although not shown, stabilizing members 650 may include one or more eyelets to assist with connecting (e.g., by sutures) the stabilizing members 650 to the occluder 600.

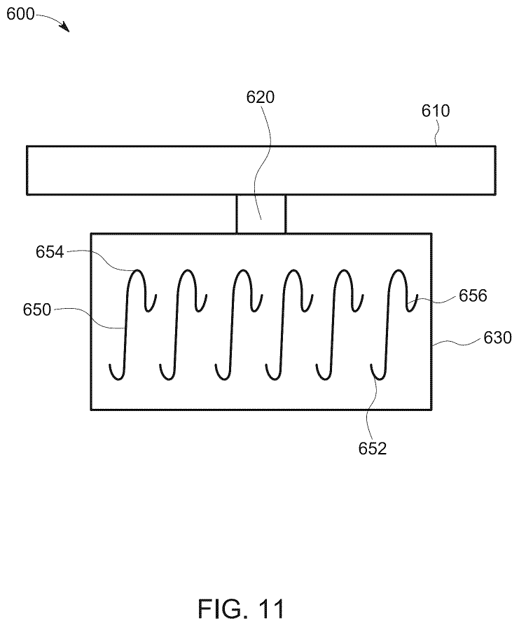

FIG. 12A is another example of an occluder with two rows of engagement features of stabilizing members at different axial positions. In this embodiment, the stabilizing members may each have a pair of engagement features (e.g. hooks or hooked ends) positioned relatively distally, with an individual engagement feature (e.g. hook or hooked end) extending from the center portion of the backing portion so that individual engagement features is positioned proximal to the pair of engagement features. In this example, occluder 700 may be substantially similar or identical to occluder 200 with the exception of the stabilizing members. For example, occluder 700 may include a proximal disk 710 connected to a distal lobe 730 by a connection portion 720. In some examples, each stabilizing member 750 may include a backing portion 754, two distal engagement features (e.g. distal hooks) 752 configured to engage tissue, and a single proximal engagement feature (e.g. proximal hook) 756 extending from the backing portion 754 and generally centered between the legs that extend to the distal engagement features 752. As should be understood, the relationship between the position of the distal engagement features 752 and the proximal engagement feature 756 may be similar or identical to the engagement features of the stabilizing members 240 in the first group of occluder 200 and the engagement features of the stabilizing members 250 in the second group of occluder 200.

FIGS. 12B-12C show examples of the stabilizing members that may achieve this three-engagement-feature embodiment. For example, FIG. 12B shows one example of a stabilizing member 750a that may be used as one or more of the stabilizing members 750. As shown in FIG. 12B, the stabilizing member 750a include a backing portion 754a that may include a connector 755a. In the illustrated example, connector 755a may take the form of two prongs extending transverse to the backing portion 754a, with the two prongs extending substantially parallel to each other with a space therebetween to define a recess, although in other embodiments the connector 755a may take other forms. Two legs may each extend from the backing portion 754a to an eyelet 757a and then to a distal engagement feature (e.g. distal hook) 752a, for example similar to as shown in FIGS. 7A-7B. Stabilizing member 750a may also include a separately formed proximal engagement feature (e.g. proximal hook 756a) that is coupled to the backing portion 754a. For example, the proximal engagement feature 756a may include its own eyelet 758a and its own connector 759a, the eyelet 758a being positioned between the connector 759a and the tip of the proximal engagement feature 756a. In the illustrated example, the connector 759a may have a generally complementary shape to connector 755a. For example, the connector 759a may take the form of two prongs extending away from the eyelet 758a, with the two prongs extending substantially parallel to each other with a space therebetween to define a recess, although in other embodiments the connector 759a may take other forms. The pair of prongs of one or both of connectors 755a, 759a may be angled slightly toward each other near the free ends thereof. The two connectors 755a, 759a may be coupled to each other so that a portion of the backing member 754a is received within the recess between the prongs of connector 759a, with a portion of connector 759a received within the recess between the prongs of connector 755a. If one or both pairs of prongs are provided with a taper or angle, a clamping force may result upon coupling the connectors 755a, 759a to help the construct remain connected, although alternatively or in addition, sutures or other members may be used (e.g. including using eyelet 758a) to help the connectors 755a, 759a to remain coupled to each other.

Whereas the stabilizing member 750a of FIG. 12B includes an interlocking proximal engagement feature (e.g. proximal hook) 756a, in other embodiments, stabilizing member 750 may be formed as a single unitary member, such as a single laser-cut stabilizing member 750b as shown in FIG. 12C. Stabilizing member 750b includes the same main component as stabilizing member 750a, such as two legs each extending from a backing portion 754b to an eyelet 757b and then to a distal engagement feature (e.g. distal hook) 752b, for example similar to as shown in FIGS. 7A-7B. Stabilizing member 750b may also include a integrally formed proximal engagement feature (e.g. proximal hook) 756b that extends from the backing portion 754b. In some examples, the proximal engagement feature 756b may include its own eyelet 758b. In other words, stabilizing member 750b may be substantially identical to stabilizing member 750a, except that stabilizing member 750b omits the connectors because stabilizing member 750b may be formed as a single integral member.

Whether stabilizing members 750 take the form shown in FIG. 12B, FIG. 12C, or another form, the resulting configuration may be such that the proximal row of engagement features (e.g. proximal hooks) has one half of the total number of distal engagement features (e.g. distal hooks), unlike other embodiments described herein. Also, unlike certain other embodiments described herein, the inclusion of both a proximal engagement feature and distal engagement features on a single assembled or formed stabilizing member may result in some amount of dependency between the hooks. For example, anchoring of the proximal (or distal) engagement features into tissue may affect the ability of the other engagement features on the same assembled or formed stabilizing member to anchor into tissue. Further, as with certain other embodiments described herein, fewer total stabilizing members may be required when providing both proximal and distal engagement features on the same stabilizing member, which may reduce complexity compared to embodiments that require proximal and distal engagement features to be positioned on separate stabilizing members. It should be understood that, in some embodiments, the position of the proximal engagement members relative to the distal engagement members in the axial direction and relative to the length of the lobe 730 is substantially the same as in other embodiments, such as that shown and described in connection with FIG. 6. However, in some embodiments, the gaps between proximal engagement features 756 and distal engagement features 752 may be even larger than in other embodiments. For example, in one embodiment in which the axial length of the lobe 730 is about 7.5 mm, the proximal engagement features 756 are positioned about 2.5 mm distally (e.g. about ⅓ or 33%) of the proximal face of the lobe 730.

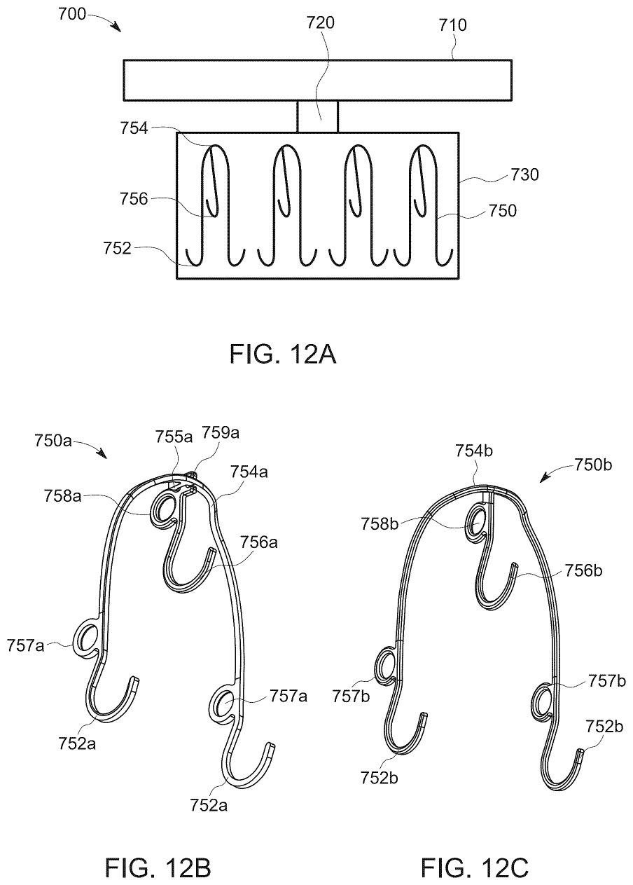

FIG. 13 is another example of an occluder with two rows of engagement features of stabilizing members at different axial positions. This example is similar to that shown in FIGS. 5-6, but in this case all of the stabilizing members have the smaller configuration (e.g., similar to the group of stabilizing members 250), with the stabilizing members positioned in two rows-one relatively distal and one relatively proximal. In this example, occluder 800 may be substantially similar or identical to occluder 200 with the exception of at least some of the stabilizing members. For example, occluder 800 may include a proximal disk 810 connected to a distal lobe 830 by a connection portion 820. Occluder 800 may include a first group of stabilizing members 840 and a second group of stabilizing members 850. Generally, the first group of stabilizing members 840, which include relatively distally positioned engagement features (e.g. hooks), may correspond to the first group of stabilizing members 240 of occluder 200, whereas the second group of stabilizing members 850, which include relatively proximally positioned engagement features (e.g. hooks), may correspond to the second group of stabilizing members 250 of occluder 200. In some examples, stabilizing members 850 may be similar or identical to stabilizing members 250, including being formed as a relatively small stabilizing member, such as the example shown and described in connection with FIG. 7A. However, unlike the relatively long stabilizing members 240 of occluder 200, the stabilizing members 840 of occluder 800 may also be relatively small stabilizing members. For example, stabilizing members 840 may be similar or identical in structure to stabilizing members 850, and only different in their relative positions on the lobe 830. The positioning of the proximal stabilizing members 850 relative to the length of the lobe 830 and relative to the distal stabilizing members 840 may be substantially similar or identical as described in connection with occluder 200.

Although many of the embodiments described herein include stabilizing members that include two groups of engagements features (e.g. hooks or hooked ends) (including individual stabilizing members with two axially offset engagement features, or separate stabilizing members being positioned so that engagement features are axially offset), it should be understood that additional stabilizing members and/or engagement features may be provided beyond the two groups. For example, any of the embodiments described herein may include additional stabilizing members and/or additional engagement features on the stabilizing members already provided so that there are three or more (e.g. four, five, six or more) groups of engagement features provided at different axial locations on the occluder.

In some examples, an occluder may be provided with stabilizing members (and/or engagement features) with a pattern that is dependent, at least in part, on the size of the device. For example, for an occluder that includes a lobe and a disk that is intended for implantation into the LAA, the occluder may be offered in eight different sizes (although more or fewer sizes may be suitable). In one example, an occluder may be offered in two size groups, including a “small” and “large” size group. For example, a “small” size group may include a lobe that has a length of about 7.5 mm and a “large” size group may include a lobe that has a length of about 10 mm. In some examples, the “small” size group may include four sizes with respective lobe and disk diameters of (1) about 16 mm and about 22 mm, (2) about 18 mm and about 24 mm, (3) about 20 mm and about 26 mm, and (4) about 22 mm and about 28 mm. In some examples, the “large” size group may include four sizes with respective lobe and disk diameters of (5) about 25 mm and about 32 mm, (6) about 28 mm and about 35 mm, (7) about 31 mm and about 38 mm, and (8) about 34 mm and about 41 mm. The different sizes of the occluder, including for example the different lengths of the lobes of the occluders, may make it desirable to have a different stabilizing member (and/or engagement feature) configuration depending on the length of the lobe. It should be understood that all dimensions provided in this paragraph are based on the occluder in the absence of applied forces (e.g. in the natural or set-shape).

As an example of the above point that different size occluder devices may be provided with different configurations of stabilizing members (and/or engagement features), a “small” size LAA occluder, with “small” being defined as a relatively small length (e.g. about 7.5 mm) lobe, may be provided with two groups of stabilizing members similar to that shown in FIG. 6. In this type of example, the occluder 200 may include a first row or group of stabilizing members 240 that are relatively large compared to a second row or group of stabilizing members 250. In this example, the engagement features (e.g. hooks or hooked ends), or the terminal ends or tips thereof, of the smaller stabilizing members 250 in the second group may be positioned between about 3.3 mm to about 4.4 mm, including about 3.8 mm, distal of the proximal plane or surface of the lobe 230 (e.g. the distance labeled “A” in FIG. 6). In this example, the engagement features (e.g. hooks or hooked ends), or the terminal ends or tips thereof, of the larger stabilizing members 240 in the first group may be positioned between about 0 mm and about 2 mm, including about 1 mm, proximal of the distal plane of surface of the lobe 230. However, for a “large” size LAA occluder that has a larger length lobe (e.g. about 10 mm compared to about 7.5 mm), it may be useful to provide stabilizing members in additional and/or different positions relative to the “small” size LAA occluder, which may be useful for providing anchoring or stabilization at additional locations. In other words, because “large” occluders are intended for use in patients with larger LAA anatomies, additional securement may be desirable (compared to “small” occluders that are intended for use in patients with smaller LAA anatomies) to account for the increased amount of tissue that the “large” occluder may need to contact and/or traverse. Providing staggered positioning of the stabilizing members, and particularly staggering the axial position of the engagement features (e.g. hooks or hooked ends) of the stabilizing members, may provide another benefit independent of that which is described immediately above. In some examples of use, the occluder (e.g. occluder 200) may need to be pulled back into the delivery device (e.g. delivery catheter 104) for repositioning after the occluder has been partially deployed (e.g. while the lobe 230 has been deployed but while the disk 210 is still coupled to the delivery device, such as to delivery cable 108). If resheathing is performed after the engagement features (e.g. hooks or hooked ends) of the stabilizing members have already deployed from the delivery device, the engagement features of the stabilizing wires may “catch” or “grab” onto the distal end of the delivery catheter 104, increasing the amount of force needed to fully resheath the occluder 200 into the delivery catheter 104. However, if the axial position of these engagement members is staggered, fewer engagement members will simultaneously “catch” or “grab” onto the distal end of the delivery catheter 104 during resheathing at any one particular time, which may reduce the force and/or difficulty required to fully resheath the occluder 200.

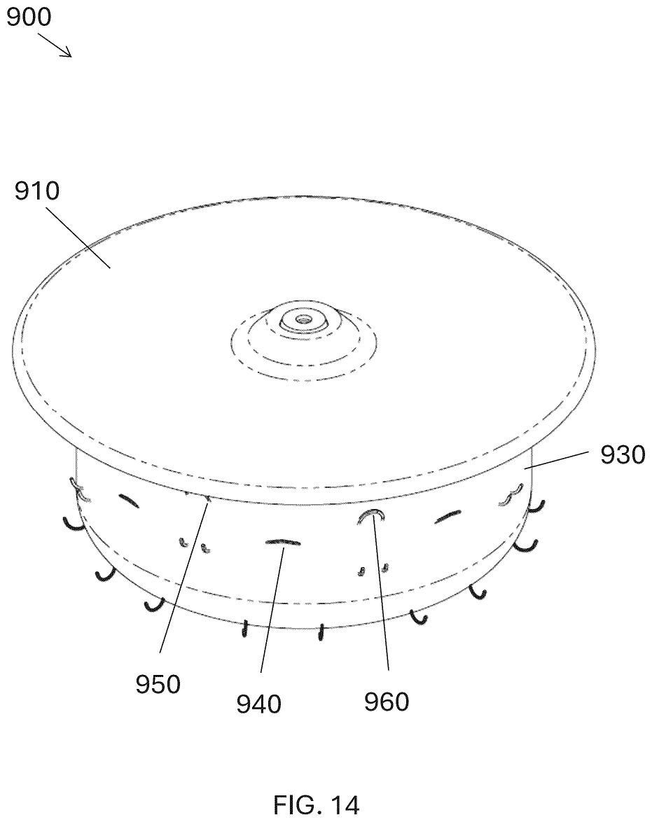

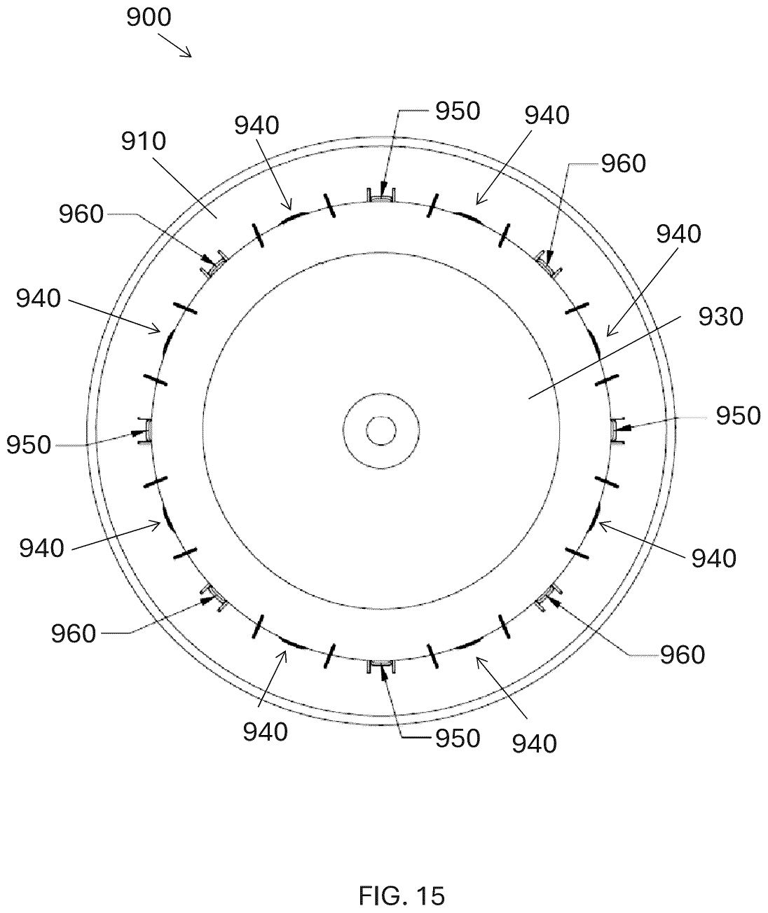

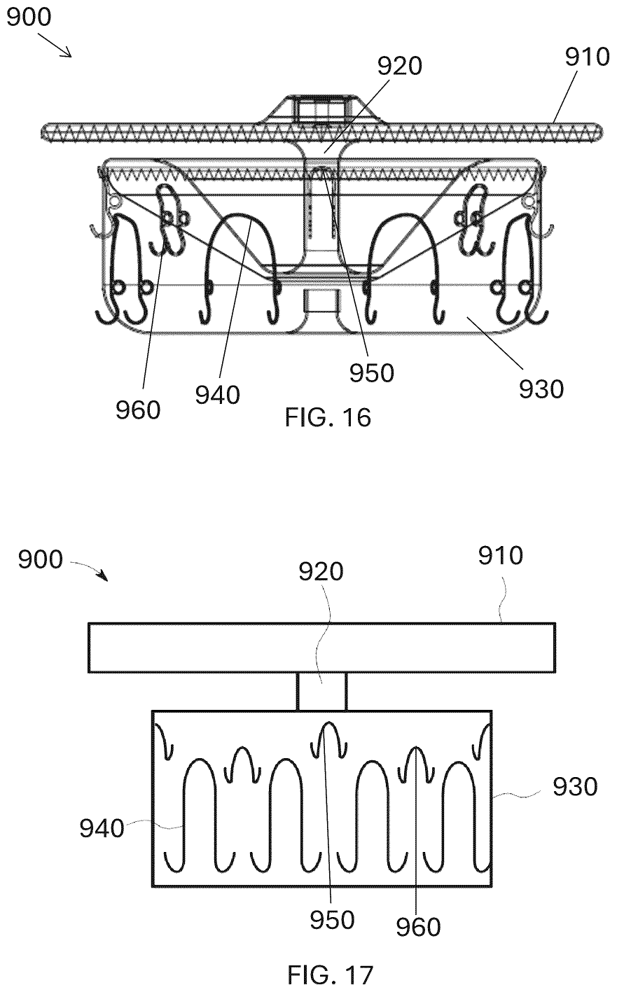

One example of an occluder 900 for use in the LAA is shown in FIGS. 14-17. Occluder 900 may be substantially similar or identical to other occluders described herein, including for example occluder 200, with exceptions regarding the positions of the stabilizing members (and/or engagement features) and/or the length of the lobe. FIGS. 14-15 illustrate top perspective, and bottom views of occluder 900, with the braided mesh forming the disk 910 and the lobe 930 depicted as a solid surface to better illustrate the positions of the various stabilizing members (and/or engagement features). FIG. 16 is a more detailed side view of the occluder 900, while FIG. 17 provides a simplified view of the occluder 900.

Referring to FIGS. 14-17, occluder 900 may be a “large” size occluder as described above, and include a proximal disk 910 connected to a distal lobe 930, for example via a connecting portion 920 (which is visible in the view of FIG. 16-17). As noted above, both disk 910 and lobe 930 may be formed of a braided mesh (e.g. strands of nitinol braided together), but the disk 910 and lobe 930 are shown as solid in FIGS. 14-15 for clarity of illustration. In this example, three separate groups of stabilizing members are coupled to the lobe 930. In the particular illustrated example, lobe 930 includes a first group of stabilizing members 940 which may be similar or identical to the first group of stabilizing members 240. For example, each stabilizing member 940 in the first group may have the same basic shape or configuration as shown in FIG. 7A or FIG. 7B, with a proximal backing portion of the stabilizing member 940 being positioned radially outward of the lobe 930, two legs of the stabilizing member 940 extending distally radially inwardly of the lobe 930, and two engagement features (e.g. hooks or hooked ends) passing through the lobe 930 to be positioned radially outward of the lobe 930 and hooking back in the proximal direction toward the disk 910. In some examples, the tips of the stabilizing members 940 (e.g. tips of the engagement features, which may be hooks) are positioned between about 0 mm and about 2 mm, including about 1 mm, proximal to the distal face of the lobe 930. In the illustrated embodiment, every other stabilizing member on the lobe 930 is a part of the first group of stabilizing members 940.

In the particular illustrated example of occluder 900, lobe 930 includes a second group of stabilizing members 950 which may be similar or identical to the second group of stabilizing members 250. For example, each stabilizing member 950 in the second group may have the same basic shape or configuration as shown in FIG. 7A or FIG. 7B, with a proximal backing portion of the stabilizing member 950 being positioned radially outward of the lobe 930, two legs of the stabilizing member 950 extending distally radially inwardly of the lobe 930, and two engagement features (e.g. hooks or hooked ends) passing through the lobe 930 to be positioned radially outward of the lobe 930 and hooking back in the proximal direction toward the disk 910. In some examples, the tips of the stabilizing members 950 (e.g. the tips of the engagement features, which may be hooks) are positioned between about 3.3 mm and about 4.3 mm, including about 3.8 mm, distal to the proximal face of the lobe 930. In the illustrated embodiment, every fourth stabilizing member on the lobe 930 is a part of the second group of stabilizing members 950.

In the particular illustrated example of occluder 900, lobe 930 includes a third group of stabilizing members 960 which may be similar or identical to the second group of stabilizing members 250, albeit with different positioning as described below. For example, each stabilizing member 960 in the third group may have the same basic shape or configuration as shown in FIG. 7A or FIG. 7B, with a proximal backing portion of the stabilizing member 960 being positioned radially outward of the lobe 930, two legs of the stabilizing member 960 extending distally radially inwardly of the lobe 930, and two engagement features (e.g. hooks or hooked ends) passing through the lobe 930 to be positioned radially outward of the lobe 930 and hooking back in the proximal direction toward the disk 910. In some examples, the tips of the stabilizing members 960 (e.g. the tips of the engagement features, which may be hooks) are positioned between about 4.4 mm and about 5.4 mm, including about 4.9 mm, distal to the proximal face of the lobe 930. In the illustrated embodiment, every fourth stabilizing member on the lobe 930 is a part of the third group of stabilizing members 960.

As best shown in FIGS. 16-17, with the particular configuration described above, the lobe 930 includes larger stabilizing members 940 alternating with smaller stabilizing members around the circumference of the lobe 930, with the smaller stabilizing members themselves alternating between a stabilizing member 950 from the second group alternating with a stabilizing member 960 from the third group, such that the order of stabilizing members around the circumference of the lobe is stabilizing member 940, stabilizing member 950, stabilizing member 940, stabilizing member 960, stabilizing member 940, stabilizing member 950 and so forth. With this configuration, the positions of the tips of the stabilizing members 940, 950, 960 (e.g. the tips of the engagement features, which may be hooks) are more varied along the length of the lobe 930 to increase the likelihood of getting good frictional engagement between the various stabilizing members 940, 950, 960 and the tissue of the LAA. This may be desirable given the large length of the lobe 930 that incorporates the three different groups of stabilizing members 940, 950, 960. Further, as noted above, during a resheathing operation, the engagement features (e.g. hooks) of stabilizing members 950 may be the first group to contact the distal end of the delivery catheter 104, and after that the engagement features (e.g. hooks) of stabilizing members 960 are the second group to contact the distal end of the delivery catheter 104, and finally the engagement features (e.g. hooks) of stabilizing members 940 are the third and final group to contact the distal end of the delivery catheter 104. This may reduce the force the user needs to create to complete recapture compared to an embodiment in which the engagement features of stabilizing members 950 and 960 all contact the distal end of the delivery catheter 104 simultaneously during resheathing. For occluders with lobes having smaller lengths, the third group of stabilizing members 960 may be replaced with the second group of stabilizing members 950, resulting in the configuration shown in, and described in connection with, FIGS. 5-6. It should also be understood that, in the embodiment of FIGS. 14-17, the proximal ends of each group of stabilizing members 940, 950, 960 are not positioned at the same axial distance as any other of the groups of stabilizing members, which may help to create less packing in these regions during compression of the occluder 900 within a delivery device.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A medical device for occluding a left atrial appendage (“LAA”), the medical device comprising:
a proximal disk configured to cover an ostium of the LAA in an implanted condition of the medical device;
a distal lobe configured to be received within a cavity of the LAA in the implanted condition of the medical device;
a connecting member connecting the proximal disk to the distal lobe;
a first group of stabilizing members, each stabilizing member in the first group having a first engagement feature configured to engage tissue surrounding the LAA in the implanted condition of the medical device; and
a second group of stabilizing members, each stabilizing member in the second group having a second engagement feature configured to engage tissue surrounding the LAA in the implanted condition of the medical device,
wherein in an unbiased condition of the medical device, the distal lobe has an axial length extending between a proximal face and a distal face of the distal lobe, the second engagement features of the stabilizing members in the second group being positioned at least 33% of the axial length beyond the proximal face of the distal lobe, the first engagement features of the stabilizing members in the first group being positioned distal to the second engagement features of the stabilizing members in the second group.

2. The medical device of claim 1, wherein the second engagement features of the stabilizing members in the second group are positioned between about 33% and about 55% of the axial length beyond the proximal face of the distal lobe.

3. The medical device of claim 1, wherein the axial length is about 7.5 mm, and the second engagement features of the stabilizing members in the second group are positioned between about 3.0 mm and about 4.0 mm beyond the proximal face of the distal lobe.

4. The medical device of claim 1, wherein the axial length is about 10 mm, and the second engagement features of the stabilizing members in the second group are positioned between about 3.75 mm and about 5.25 mm beyond the proximal face of the distal lobe.

5. The medical device of claim 1, wherein each stabilizing member in the first group is positioned between a pair of stabilizing members in the second group.

6. The medical device of claim 1, wherein each stabilizing member in the first group is substantially identical in structure to each stabilizing member in the second group.

7. The medical device of claim 1, wherein each stabilizing member in the first group has an axial length that is equal to or longer than an axial length of each stabilizing member in the second group.

8. The medical device of claim 1, wherein a total number of the stabilizing members in the first group is equal to a total number of stabilizing members in the second group.

9. The medical device of claim 1, wherein a spacing between each adjacent pair of stabilizing members is substantially equal.

10. The medical device of claim 1, wherein a radial spacing between each adjacent pair of stabilizing members is between about 20 degrees and about 65 degrees.

11. A medical device for occluding a left atrial appendage (“LAA”), the medical device comprising:
a proximal disk configured to cover an ostium of the LAA in an implanted condition of the medical device;
a distal lobe configured to be received within a cavity of the LAA in the implanted condition of the medical device;
a connecting member connecting the proximal disk to the distal lobe;
a first group of stabilizing members, each stabilizing member in the first group having a first engagement feature configured to engage tissue surrounding the LAA in the implanted condition of the medical device; and
a second group of stabilizing members, each stabilizing member in the second group having a second engagement feature configured to engage tissue surrounding the LAA in the implanted condition of the medical device; and
a third group of stabilizing members, each stabilizing member in the third group having a third engagement feature configured to engage tissue surrounding the LAA in the implanted condition of the medical device,
wherein in an unbiased condition of the medical device, the distal lobe has an axial length extending between a proximal face and a distal face of the distal lobe, the first engagement features are positioned at a first distance along the axial length, the second engagement features are positioned at a second distance along the axial length, and the third engagement features are positioned at a third distance along the axial length, the first distance being different than the second distance, the second distance being different than the third distance, the third distance being different than the first distance.

12. The medical device of claim 11, wherein the axial length is about 10 mm.

13. The medical device of claim 12, wherein the first distance is between about 8 mm and about 10 mm, such that the first engagement features are positioned between about 0 mm and about 2 mm proximally of the distal face of the distal lobe.

14. The medical device of claim 13, wherein the second distance is between about 3.3 mm and about 4.3 mm.

15. The medical device of claim 14, wherein the third distance is between about 4.4 mm and about 5.4 mm.

16. The medical device of claim 11, wherein the medical device includes a total number of stabilizing members in the first group that is twice as many as a total number of stabilizing members in the second group and that is twice as many as a total number of stabilizing members in the third group.

17. The medical device of claim 11, wherein a single stabilizing member in either the second group or the third group is positioned between each circumferentially adjacent pair of stabilizing members in the first group.

18. The medical device of claim 11, wherein each stabilizing member is separate from the other stabilizing members, wherein each group of stabilizing members comprises more than one stabilizing member, and wherein each stabilizing member is connected to the distal lobe.

19. The medical device of claim 11, wherein each stabilizing member in the first, second, and third groups is either (i) formed of a wire or (ii) cut from a sheet of material.

20. The medical device of claim 11, wherein each first engagement feature, each second engagement feature, and each third engagement feature is a hook.
